(19) United States
(12) Patent Application Publication
(10) Pub. No.: US 2015/0314075 A1
(43) Pub. Date: Nov. 5, 2015

(54) CLUTCH MEMBER FOR AN INJECTION DEVICE

(71) Applicant: OWEN MUMFORD LIMITED, Oxford (GB)

(72) Inventor: Toby COWE, Oxford (GB)

(73) Assignee: OWEN MUMFORD LIMITED, Oxford (GB)

(21) Appl. No.: 14/440,203

(22) PCT Filed: Oct. 23, 2013
(86) PCT No.: PCT/GB2013/052761

§ 371 Date: May 1, 2015

Related U.S. Application Data
(60) Provisional application No. 61/721,620, filed on Nov. 2, 2012.

(30) Foreign Application Priority Data
Nov. 2, 2012 (GB) 1219753.9

(51) Int. Cl. A61M 5/315

(52) U.S. Cl. CPC A61M 5/31536; A61M 5/31551

(57) ABSTRACT

An injection device includes a housing (10, 16) for receiving a cartridge or syringe (18), a plunger (50) moveable axially to express a dose from the cartridge or syringe, a rotary drive element (22) configured to rotate as the plunger moves axially, a clutch member (38) moveable between a holding position, in which it inhibits rotary movement of the rotary drive element (22), and a release position, in which the rotary drive element is freed to rotate to cause or allow the plunger (50) to move axially; wherein the clutch member (38) is selectively moveable to a control position to control rotary movement of the rotary drive element and thereby axial movement of the plunger, and bias element (94) for urging the clutch member to its control position.

This invention relates to injection devices and, in particular but not exclusively, to injection devices for injecting medicament in multiple doses of an adjustable magnitude.

In our earlier Autopen® device, a rotatable dose setting knob at the rear end of a pen injector is connected to a hollow drive sleeve that carries a circumferentially extending ratchet arm at its front end which engages a corresponding ratchet surface on a threaded drive collar. The threaded drive collar is threadedly engaged with the stem of a plunger, so that a rotary movement of the drive collar is converted into a linear advance of the plunger. A trigger can be moved to hold or release the drive collar for rotary movement. During a dose setting routine, with the drive collar held against rotation by the trigger, a selectable dose increment is dialed in by rotating the dose setting knob and the drive sleeve away from a ‘0’ position against a spring bias afforded by a main torsion drive spring. The dose setting movement is unidirectional only, due to ratchet action between the ratchet arm on the drive sleeve and the drive collar. Upon release of the trigger, the drive sleeve and the drive collar rotate as one by an amount equivalent to the angular dose increment dialed in, with the plunger being advanced by the corresponding amount to deliver the required magnitude of dose.

Although the device performs extremely well and enjoys considerable success, we have determined that it is desirable to provide the user with a degree of control over the delivery of the medicament. The degree of control may be to reduce the speed of delivery of the medicament (particularly where the medicament is of high viscosity), or to allow the user to interrupt a dose delivery to allow the user to effect delivery of the dose in small steps.

Our co-pending WO2011/045611 discloses an alternative type of arrangement for allowing a user to set and then initiate medicament delivery, and again we have found that a need exists for allowing the user to control the delivery cycle.

Accordingly, in one aspect, this invention provides an injection device comprising:

a housing for receiving a cartridge or syringe;

a plunger moveable axially in use to express a dose from said cartridge or syringe;

a rotary drive element configured to rotate as said plunger moves axially;

a clutch member moveable between a holding position, in which it inhibits rotary movement of said rotary drive element, and a release position, in which the rotary drive element is freed to rotate to cause or allow said plunger to move axially;

wherein said clutch member is selectively moveable to a control position to control rotary movement of said rotary drive element and thereby axial movement of said plunger, and

bias means for urging said clutch member to its control position.

In various embodiments, as the plunger moves axially to express a dose, a rotary drive element rotates correspondingly. It is envisaged that, in some arrangements, the rotary drive element is driven and rotates to impart linear movement to the plunger, whilst in other arrangements, a stored energy device may impart a linear movement to the plunger which causes corresponding rotary movement of the rotary drive element. In either instance, the clutch member may be disengaged at the beginning of a dose expression cycle and then reengaged as required to provide a braking or interruption effect.

The clutch member may take many forms, with cooperating engagement surfaces provided on the clutch member and the rotary drive element or an associated element. Biasing the clutch member to its control position means that the expression cycle may be slowed or interrupted by default if pressure is released from the clutch member.

In some embodiments the control position is the same as or adjacent to the holding position, although we do not exclude arrangements in which the control position is spaced away from the holding position either between the holding and release positions or beyond the release position.

Conveniently, the clutch member is moved directly or indirectly by means of an externally accessible trigger element which is manually operable to move the clutch member between the holding position, the release position and the control position.

Although other types of movement are possible, for example rotary, hinging or lateral deflection, it is preferred for the trigger element to be moveable axially to effect operation of said clutch member.

Conveniently, said trigger element is also configured to be moveable to set an adjustable extent of expression movement of said plunger when released, thereby to set a dose magnitude. The trigger element may itself directly set an extent of movement of the plunger but in many instances, there will be one or more additional co-operating components associated with the plunger or the trigger element which ensure a dose setting selection of said trigger element results in a corresponding extent of expression movement of the plunger when released. Although other types of movement are possible, it is preferred for the trigger element to be moveable angularly to set said dose.

Conveniently a scale member is carried for linear movement relative to said rotary drive element but is secured against rotation therewith. In some arrangements said scale member may be threadedly mounted for rotation with respect to said trigger element, with there being a stop limiting the extent of rotary threaded movement in at least one direction. Thus in one example the scale member is of tubular form, having an inner surface engaging said rotary drive element in a manner allowing axial sliding movement but preventing rotation between the two. An outer surface may be provided with a feature that threadedly engages a feature disposed in an internal bore provided in the trigger element. The outer surface may carry indicia representing dose amounts that can be read off a marker or window on the trigger element.

Preferably said clutch member is mounted for axial sliding movement relative to said housing but is constrained against rotation with respect thereto. Preferably said clutch member and said rotary drive element have cooperating features that engage when the clutch is engaged. Preferably, the features are disposed so that the clutch may be engaged in a plurality of different angular positions.

Preferably, the trigger element is configured to be moveable from a dose-setting position to a firing position which correspondingly causes movement of said clutch member from its holding position to its release position. When in its dose-setting position, the trigger element is preferably rotatable to set a dose. Conveniently, said trigger member is configured so that, as it moves towards said firing position, rotary movement of said trigger member is inhibited. This may be achieved by means of corresponding features on the trigger element and on said housing engaging axially in a rotation prevention position as said trigger member approaches or reaches its firing position.

Conveniently, the rotary drive element may carry splines or other projections that extend axially to cooperate with grooves in the scale member (where provided) and also to cooperate with inwardly projecting teeth or other elements provided on the clutch member.

In another aspect, this invention provides an injection device comprising:

a housing for receiving a cartridge or syringe;

a plunger moveable axially to express a dose from said cartridge or syringe;

a rotary drive element configured to rotate as said plunger moves axially;

a dose setting member biased towards a dose setting position in which it is rotatable in a dose setting step to set a selectable angular dose increment;

a clutch member configured to inhibit rotational movement of said rotary drive element during dose setting movement of said dose setting member;

wherein, having set a dose, the dose setting member is movable against said bias to a release position to effect release of said drive member to allow it to rotate by an amount dependent on the selected angular dose increment, thereby to allow or cause said plunger to move axially by a corresponding amount.

In some applications it is desirable to provide the user with contemporaneous feedback of the progress of the expulsion phase of the device, as a dose is delivered. In some embodiments, the injection device may be equipped with an arrangement that emits an audible and/or tactile confidence signal. Preferably the confidence signal is generated mechanically, for example by a clicker action or the like whereby a series of clicks or impacts are generated consequent on movement of the plunger. This may be generated directly by the plunger, for example by means of mechanical interaction between the plunger and cooperating element or surface, or it may be generated indirectly by an element that moves as the plunger moves. Thus where, as above, the plunger is caused to move by rotary movement of a rotary drive element, the rotary element and an associated element or surface may interact to provide the confidence signal. Equally, any other element that moves as the plunger moves may be used in a similar manner.

The signal is conveniently generated by means of a resiliently moveable clicker element that moves along an path interrupted by spaced abutments, or a notched or serrated track, which may be linear or circular, so that a series of clicks is emitted as the respective movement occurs. Conveniently the clutch element may be provided on the drive shaft and cooperating with a path or track provided on an element that remains fixed as the drive shaft rotates. Thus the track may be provided on the housing of the device, or, for example, on a firing element or trigger element. It will of course be appreciated that the clicker element could instead be provided on the relatively stationary component whilst the path or track is provided on the rotating element.

In another aspect this invention provides an injection device comprising:

a housing receiving a cartridge or syringe;

a plunger moveable axially to express a dose from said cartridge or syringe;

an arrangement for emitting a series of mechanically generated tactile and/or audible signals during at least part of the movement of said plunger.

Whilst the invention has been described above, it extends to any inventive combination or sub-combination of the features set out above or in the following description, drawings or claims.

The invention may be performed in various ways and, by way of example only, various embodiments thereof will be described in detail, reference being made to the accompanying drawings, in which:

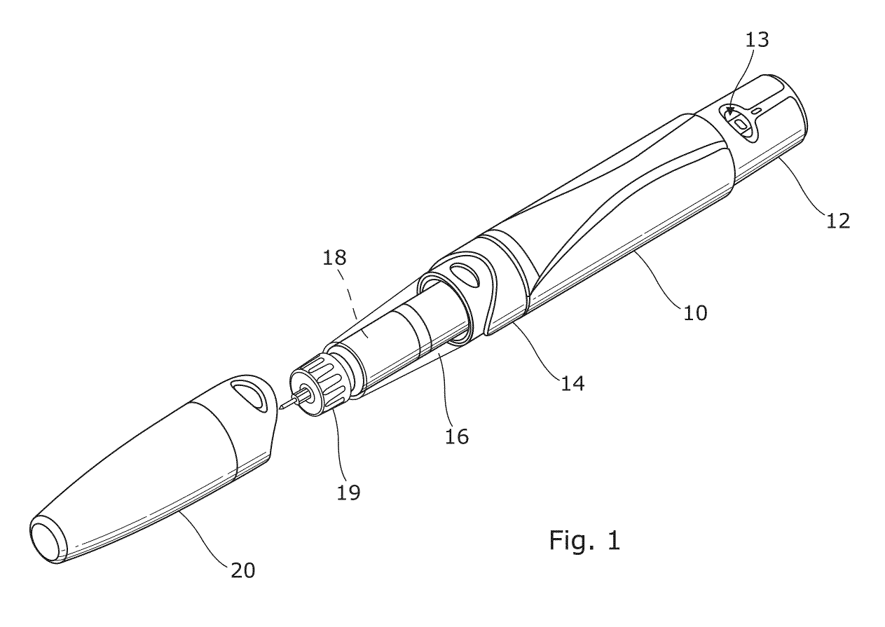

FIG. 1 is an exploded view of an injection device in accordance with the first embodiment of this invention;

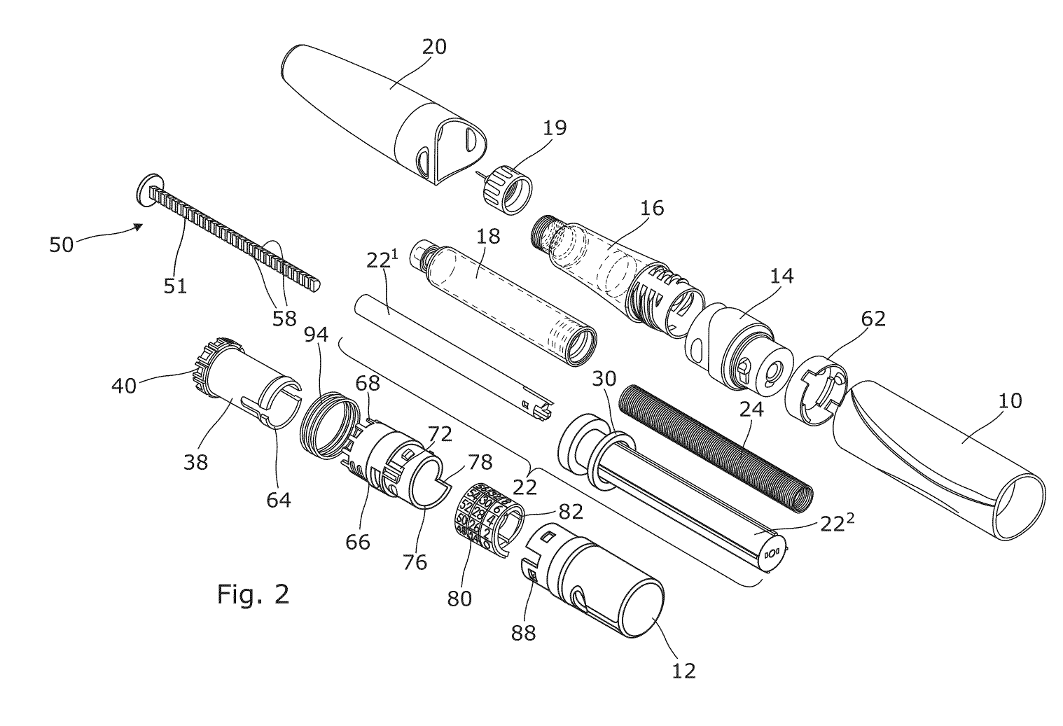

FIG. 2 is a side view of the device in FIG. 1, when assembled;

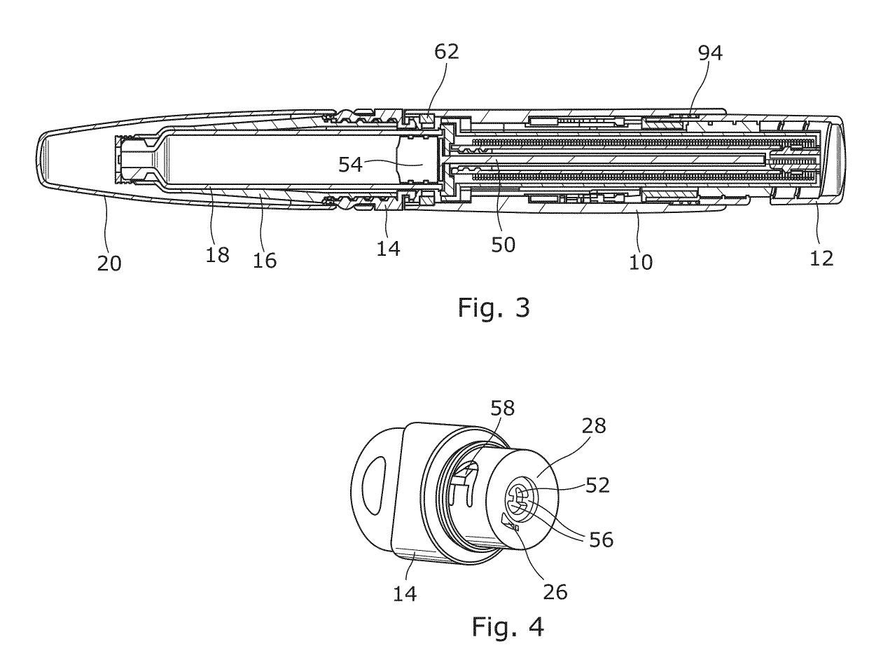

FIG. 3 is a longitudinal cross-section view through the arrangement of FIG. 2;

FIG. 4 is a detailed rear view of the rewind collar of the first embodiment;

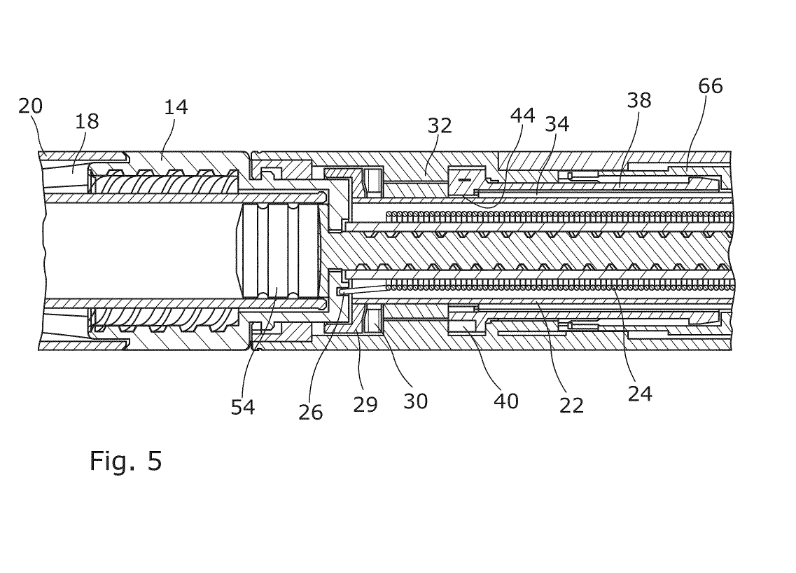

FIG. 5 is a detailed cross section view of the mid-region of the first embodiment, showing the forward end of the drive shaft, the clutch and the rewind collar;

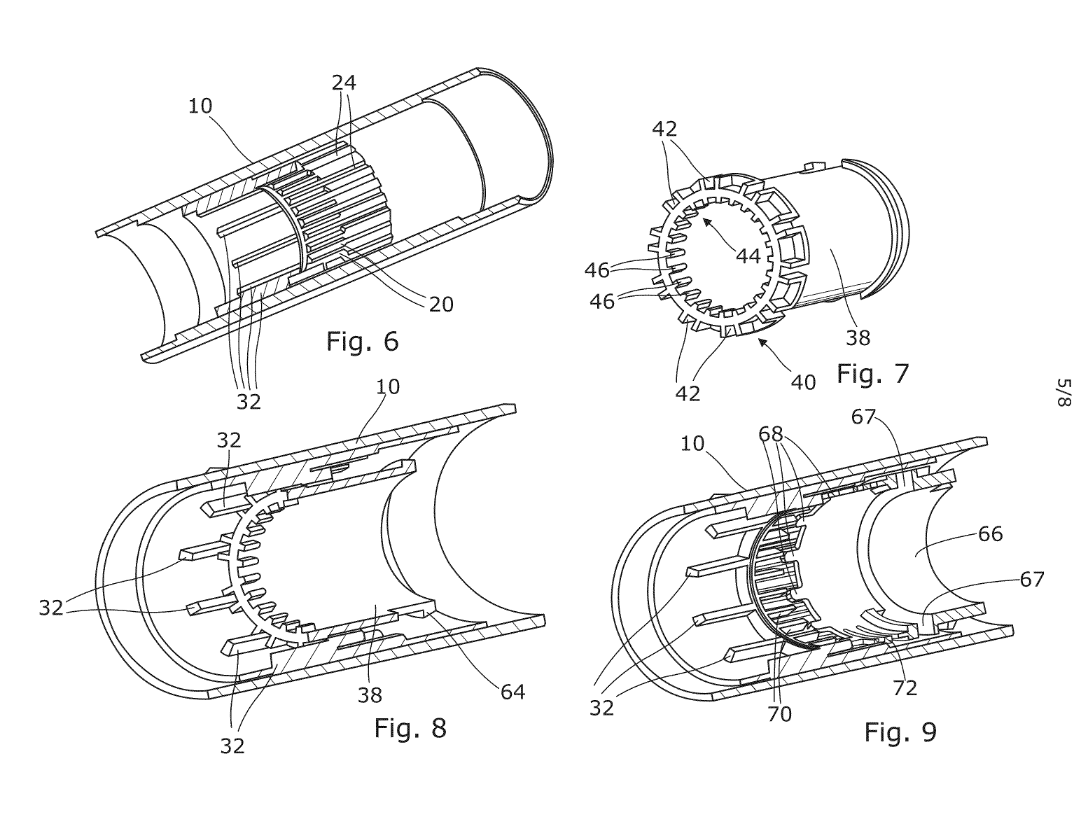

FIG. 6 is a detailed view of the inner surface of the body of the first embodiment showing the formations provided on the inner surface thereof;

FIG. 7 is a detailed front perspective view of the clutch member of the first embodiment;

FIG. 8 is a detailed section view showing the engagements between the clutch and the formations on the inner wall on the inner body of the first embodiment;

FIG. 9 is a view showing the inter-engagement between the dose setter and the inner body formations in the first embodiment;

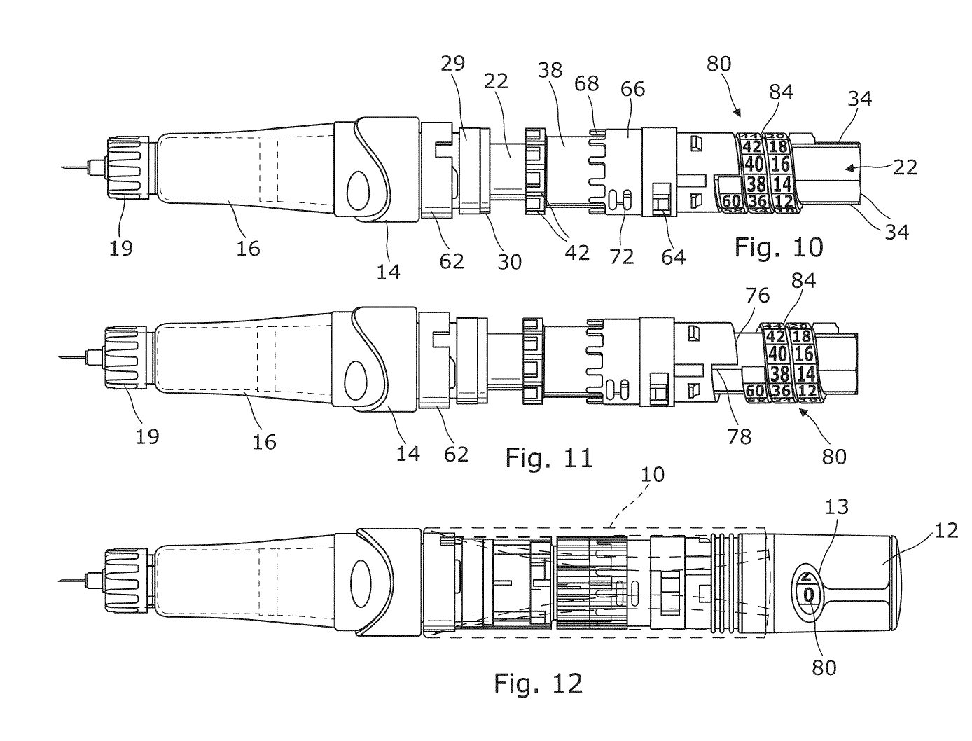

FIGS. 10 and 11 are side views of the first embodiment but with the body and trigger button removed, and showing the scale in different positions.

FIG. 12 is a view similar to FIGS. 10 and 11 but showing the body and trigger member in position, with the body shown in phantom;

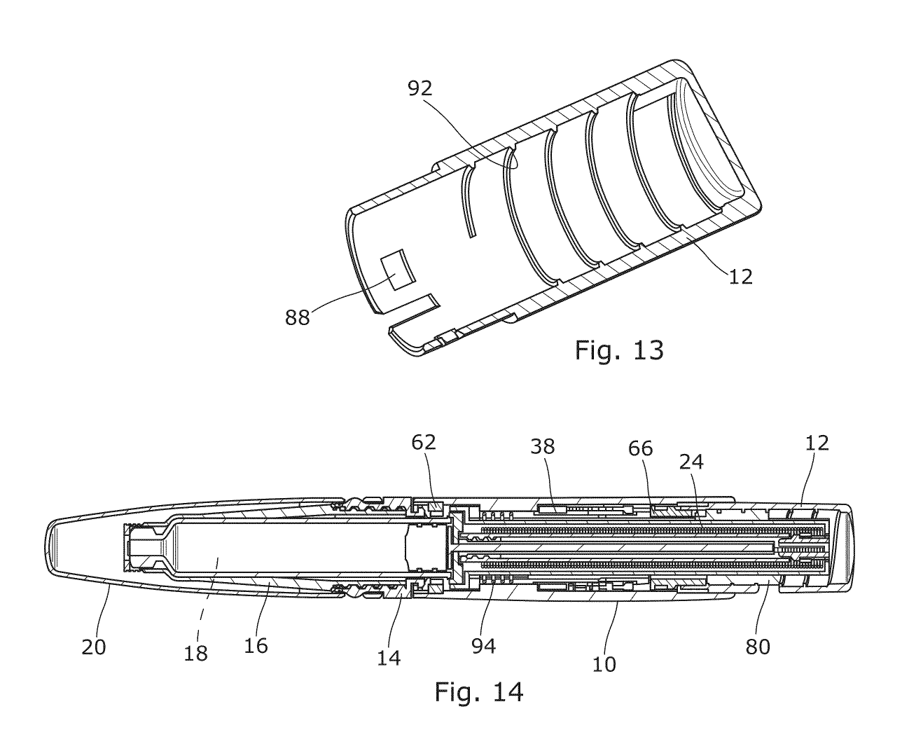

FIG. 13 is a detailed section view showing the inside of the trigger member;

FIG. 14 is a longitudinal section view of a second embodiment of injection device, showing an alternative location for a bias spring, and

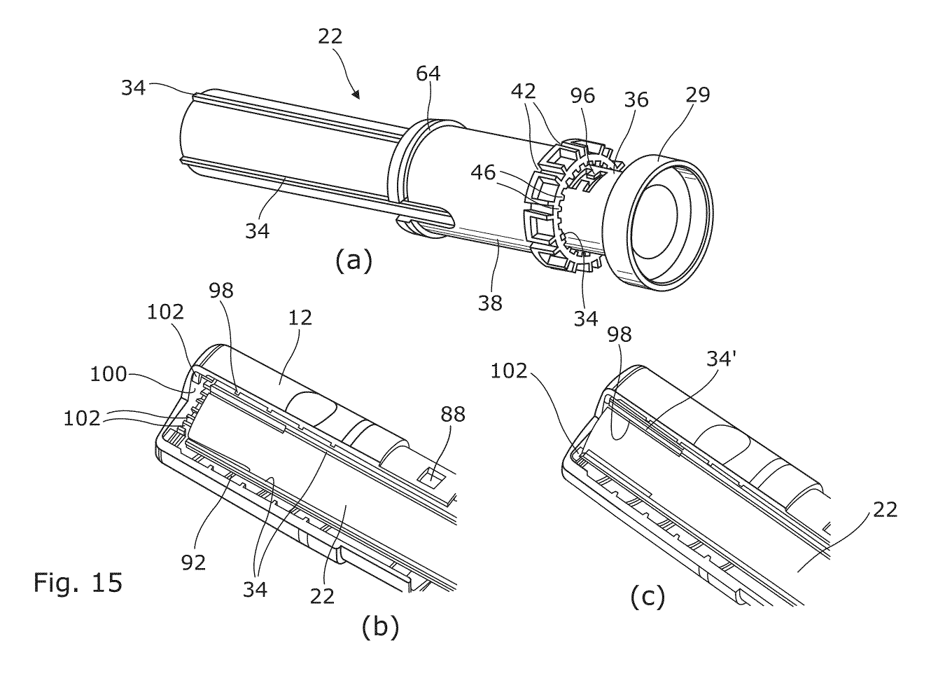

FIGS. 15 (a), (b) and (c) are detail views of two alternative clicker arrangements for sounding as the medicament is expelled to deliver a dose.

Referring initially to FIG. 1, the embodiment of injection device described herein is a pen-type injector intended to allow a user to dial in a number of units to make up a selected dose magnitude, offer up the injection device to the skin, manually insert the needle to the correct depth and then fire the device to cause the dose of selected magnitude to be introduced into the injection site through the needle. The injection device comprises a main body 10 containing the dose setting and driving mechanisms, a trigger button 12, a rewind collar 14 into which is screwed a cartridge housing 16 containing a cartridge 18. The trigger button 12 is rotatable to allow a dose to be dialed in before the device is fired. The cartridge housing is threaded at its forward end to allow a disposable double-ended needle tip 19 to be screwed onto the cartridge housing so that the contents of the cartridge may be expelled through the forward end of the needle tip. A removable safety cap 20 releasably clips over the cartridge housing, clipping on to the rewind collar.

The device is shown in exploded form in FIG. 2. A drive shaft 22 comprises an inner drive shaft portion 22¹ and an outer drive shaft portion 22². During assembly, the inner drive shaft 22¹ is fitted inside the outer drive shaft with the two being permanently attached so that they rotate and move axially together. Although as shown the drive shaft is assembled from two portions, in other embodiments the drive shaft may be of unitary construction. In the annular space defined between the inner and outer drive shafts is disposed a torsion spring 24 with the rear end of the torsion spring being angularly fixed to the inner drive shaft 22² by suitable means. The forward end of the torsion spring 24 is secured to a recess 26 provided in a rear flat wall 28 of the rewind collar 14, which can be seen in FIGS. 4 and 5. Referring more particularly to FIG. 5 the drive shaft 22 is mounted in the body 10 to allow rotation but not axial movement thereof. As seen in FIG. 5, the forward end 29 of the outer drive shaft 22² abuts the flat wall 28 of the rewind collar 14. This prevents forward axial movement of the drive shaft. Rearward movement of the drive shaft is prevented by means of a washer 30 that abuts on one side a flat, rearward facing portion 31 of the outer drive shaft and at its rearward surface locates against the forward ends of a number of splines 32 that project inwardly from the inner cylindrical wall of the body 10, the internal splined area of the body 10 area being shown in detail in FIG. 6.

The outer drive shaft 22² has four longitudinal ribs 34 equispaced about its periphery each of which extends forwardly from the rear end of the outer drive shaft but stops short of the forward end thereof to leave a plain outer cylindrical section 36.

A clutch 38, illustrated in detail in FIG. 7 is of generally cylindrical form and carries at its forward end an outer ring 40 defining a number of equispaced axially extending grooves 42. When assembled, as seen in FIG. 8, grooves in the outer ring engage the splines 32 on the body portion and this engagement ensures that the clutch can move axially within the body but is prevented from rotation. The clutch has an inner ring 44 having teeth 46 which, when the clutch is in an engaged position, fits over the longitudinal ribs 34 of the drive shaft 22 to prevent rotation thereof. The number of teeth on the inner ring is an integral multiple of the number of ribs 34 and the drive shaft to hold it in any one of a relatively large number of incrementally spaced angular positions.

The clutch 38 can be moved relative to the drive shaft between a rearward, engaged, position in which the inner teeth 46 of the clutch engage the outer ribs 34 on the drive shaft to prevent rotation thereof, and a forward, disengaged position, where the teeth 46 of the clutch are disposed forwardly of the ribs and adjacent to the plain section 36, such that rotation of the drive shaft relative to the housing under the influence of the torsion spring 24 is permitted. The manner in which the clutch member is moved between its engaged and disengaged positions will be described below.

The inner drive shaft 22¹ has a threaded inner bore which threadedly receives a threaded plunger 50. The threaded plunger 50 has a stem 51 that extends through a shaped orifice 52 in the rear end wall 28 of the rewind collar 14 to engage the piston 54 of a cartridge 18 contained in the cartridge housing (see FIGS. 4 and 5). The orifice 52 has two diametrically facing fingers 56 that engage in diametric opposed slots 58 extending down the threaded length of the plunger 50, to allow the plunger to move axially with respect to the rewind collar 14 but to prevent rotational movement with respect thereto. In use, as the drive shaft 22 is allowed to rotate under the influence of the torsion spring 26, relative to the housing (and therefore the plunger), the plunger advances axially to drive the cartridge piston 54 forwardly.

As seen in FIG. 4 the rewind collar 14 is provided on a rear cylindrical surface with two opposed circumferential ratchet teeth 58 that cooperate with appropriately shaped abutments 60 in a ratchet bush 62 to allow rotation of the rewind collar 14 in a direction to energise the torsion spring 24 when it has become partially or fully unwound. The threaded plunger 50 rotates with the rewind collar during the rewind operation causing the plunger to be retracted back into the inner drive shaft 22¹.

Referring now to FIGS. 7 and 9 to 12, the clutch 38 is provided at its rear end with a snap fitting 64 which enables the clutch to be assembled with a dose setter element 66. The dose setter element is of generally cylindrical form having an internal annular groove or recess 67 into which the snap fitting 64 of the clutch 38 fits to connect the two to move as one longitudinally movement but to allow relative rotational movement. The dose setter 66 is provided at its front end with a number of forward facing fingers 68 that, when the dose setter 66 is shifted forwardly with the clutch, fit into corresponding pockets 70 provided on the inside of the main body portion as shown in FIGS. 6 and 9 to prevent relative rotation. However, when the dose setter 66 is in a rearward position, it may rotate relative to the main body with a clicker action provided by a resilient pip 72 cooperating with fluted wall portions 74 that form extensions of the pockets 70 in the body portion.

The rearward portion of the dose setter 66 has a helical form shown generally at 76, with a stop surface shown at 78. As seen in FIGS. 10 to 12 when the device is assembled, the rear end of the drive shaft 22 passes through the dose setter 66 so that the rear end projects rearwardly thereof. Slideably received on the rear end of the drive shaft 22, is a helical cylindrical scale 80 that carries on its inner surface four longitudinal grooves 82 (see FIG. 2) which engage the ribs 34 on the drive shaft, and carries on its outer surface a helical thread groove 84. In the rest position, shown in FIG. 10 and when the torsion spring is partly or fully charged, and the clutch 38 is in its rearward position, the drive shaft 32 is held against rotation by inter engagement of the teeth 46 on the inner ring of the clutch 38 with the ribs 34 on the drive shaft. The clutch is prevented from rotating by engagement of the grooves 42 on the outer ring 40 with the splines 32 on the inside of the body portion. The scale is disposed with a stop face 86 on the forward end thereof facing the stop face 78 on the dose setter 66 as shown in FIG. 10. The trigger button 12 is of generally cylindrical form closed at its rear end and clips over the rear end of the dose setter 66 by means of apertures 88 snapping over ribs 90 on the dose setter 66 so that the dose setter and trigger button move as one. The trigger button has a helical thread provided on its inner surface (see FIG. 13) which engages the helical groove 84 on the scale member. The trigger button 12 has a window 13 through which indicia on the scale 80 can be read off.

From the rest position shown in FIG. 10, rotating the trigger button 12 in the appropriate sense causes the trigger button 12 and the dose setter 66 to rotate as one with increments determined by the click action between the pip 72 and the fluted portions 74. During this dose setting stage, none of the other components of the device rotate. Thus, the engagement between the helical thread 92 on the inside of the trigger button 12 and the helical groove 84 on the scale 80 means that the scale shifts rearwardly by an amount dependent on the angular rotation of the trigger button 12, for example to the position shown in FIG. 11. From this position if the trigger button is pushed forwardly, the dose setter 66 and clutch 38 both move forwardly, with the dose setter being held against rotation as soon as the fingers 68 on its front end enter the pockets 70 on the inside of the body (not shown in FIGS. 10 to 12). Sometime after the fingers 68 initially engage the pockets 70, continued forward movement of the trigger button 12, dose setter 66 and clutch 38 cause the clutch to move to its disengaged position where the teeth 46 on the inner ring 44 of the clutch move forwardly off the ribs 34 on the drive shaft 22 so that the drive shaft can rotate. The scale 80 rotates with the drive shaft 22 due to the engagement between the ribs 34 and the grooves 82 and the rotation of the scale 80 relative to the stationary trigger button 12 means that the scale rotates, with the stop face 86 of the scale following a helical path until it returns to the position in which it abuts the stop face 78 on the dose setter 66 preventing function rotation. The rotation of the drive shaft 22 moves the plunger 50 forward axially by a corresponding amount, to express a dose.

In this embodiment the clutch 38 is biased rearwardly to its engaged position so that, during expression of a dose, if the user releases pressure from the trigger button, delivery of the dose will be interrupted or slowed as the clutch re-engages. Reapplication of forward pressure to the trigger button will allow expression of the dose to accelerate or restart. The clutch spring 94 in this first embodiment is disposed to act between a forward facing shoulder on the trigger button 12 and a rearward facing shoulder provided on the inside of the body as seen in FIG. 3. In another embodiment, shown in FIG. 14, the spring 94 is disposed between the forward end of the clutch 38 and the washer 30 to provide a similar effect of biasing the assembly of the clutch, dose setter and trigger button rearwardly, with a similar effect on operation of the device.

In use, the user will unscrew the cartridge housing 16 and dispose of any spent cartridge 18. The drive and dosing mechanism is then recharged by rewinding the rewind collar 14 in the direction allowed by the ratchet action to charge the torsion spring and to retract the plunger into the main body. A fresh cartridge 18 is loaded into the cartridge housing and the cartridge housing screwed back on to the rewind collar. A needle may then be screwed onto the front end of the cartridge holder. For the first or for a priming operation, the user will rotate the trigger button until the required digit on the scale member is visible through the trigger button window. This setting action determines an extent of angular movement of the drive shaft when released. Pushing the trigger button into the housing moves the trigger button, dose setter and clutch forwardly. This initially engages the interlock between the dose setter and the housing to prevent angular movement of the dose setter and the trigger button during expulsion movement and thereafter releases the clutch so that the drive shaft can rotate under the influence of the drive spring by the required amount.

Referring now to FIGS. 15(a) to (c) there are shown two alternative clicker arrangements for use in either of the above embodiments and which are designed to emit a series of clicks as the drive shaft 22 rotates when released by pushing the trigger button. In the first of these arrangements, illustrated in FIG. 15(a) the drive shaft 22, in the otherwise plain region 36 forwardly of the four equispaced ribs 34, is provided with a resiliently mounted pip 96. The pip 96 is disposed so that, when the clutch 38 is moved forwardly on firing the device to move it forwardly out of engagement with the ribs 34 to allow the drive shaft to rotate, the inner teeth 46 on the clutch lightly engage the pip 96. As the released drive shaft rotates, the pip 96 emits a series of clicks due to the clicking action as it runs lightly over the teeth 46 of the clutch. It will of course be appreciated that the number of clicks gives an indication of the volume of the dose that has been expressed, and the frequency of the clicks indicates the speed of expulsion. In the event that the user releases pressure on the trigger 12 to slow or interrupt the delivery of the dose, the emission of clicks will be correspondingly slowed or interrupted.

Referring now to the arrangement shown in FIGS. 15(b) and (c), here the rearward end 34¹ of each rib 34 is cantilevered by means of a slot 98 formed at the base of each extension so that each end portion can flex resiliently in a generally tangential sense relative to the drive shaft. The facing inner end wall 100 of the trigger 12 is provided with a circumferential arrangement of equispaced upstanding ribs 102. The ribs 102 and rearward ends 34¹ are designed so that when the trigger 12 is pushed forwardly to fire the device, the upstanding ribs 102 lie in the path of the tips of the rearward ends of the ribs 34. As the drive shaft 22 rotates, the tips run lightly over the upstanding ribs 102 to provide a clicking action. As above, the number and frequency of the clicks are dependent on the magnitude and speed of delivery of the dose.

1. An injection device comprising:
a housing (10, 16) for receiving a cartridge or syringe (18);
a plunger (50) moveable axially to express a dose from said cartridge or syringe;
a rotary drive element (22) configured to rotate as said plunger moves axially;
a clutch member (38) moveable between a holding position, in which it inhibits rotary movement of said rotary drive element (22), and a release position, in which the rotary drive element is freed to rotate to cause or allow said plunger (50) to move axially;
wherein said clutch member (38) is selectively moveable to a control position to control rotary movement of said rotary drive element and thereby axial movement of said plunger, and
bias means (94) for urging said clutch member to its control position.

2. An injection device according to claim 1 wherein said control position is at or adjacent said holding position.

3. An injection device according to claim 1, wherein said clutch member (38) is operable in said control position to effect at least one of reduction of angular velocity and interruption of said rotary movement.

4. An injection device according to claim 1, including an externally accessible trigger element (12) manually operable to move said clutch member (38) between said holding position, said release position and said control position.

5. An injection device according to claim 4,
wherein said trigger element is moveable axially to effect operation of said clutch member.

6. An injection device according to claim 1 wherein said trigger element (12) is also configured to be moveable to set an adjustable extent of expression movement of said plunger (50) thereby to set a dose magnitude.

7. An injection device according to claim 6, wherein said trigger element (2) is moveable angularly to set said dose.

8. An injection device comprising:
a housing (10, 16) for receiving a cartridge or syringe (18);
a plunger (50) moveable axially to express a dose from said cartridge or syringe;
a rotary drive element (22) configured to rotate as said plunger moves axially;
a dose setting member (12) biased (94) towards a dose setting position in which it is rotatable in a dose setting step to set a selectable angular dose increment;
a lock member (38) configured to inhibit rotational movement of said rotary drive element (22) during dose setting movement of said dose setting member (22);
wherein, having set a dose, the dose setting member is movable against said bias to a release position to effect release of said lock member to allow said drive member to rotate by an amount dependent on the selected angular dose increment, thereby to allow or cause said plunger (50) to move axially by a corresponding amount.

9. An injection device according to claim 8, wherein said dose setting member is moveable to said release position by application of manual pressure, and release of said manual pressure allows return of said lock member to interrupt or modulate movement of said rotary drive element.

10. An injection device according to claim 1, including an arrangement for emitting a periodic tactile and/or audible signal during at least part of the movement of said rotary drive element.

11. An injection device according to claim 10, wherein said arrangement includes cooperating elements provided on or associated with said rotary drive element and a component that does not rotate with said rotary drive element, respectively.

12. An injection device accordingly to claim 11, wherein said cooperating elements include a resiliently mounted element cooperating with a plurality of surfaces making up a notched or uninterrupted track.

13. An injection device according to claim 11 wherein the element provided on or associated with said rotary drive element cooperates with an element provided or associated with said clutch member.

14. An injection device according to claim 11, wherein the element provided or associated with said rotary drive element cooperates with an element provided on or associated with said dose setting member.

15. An injection device according to claim 10 wherein said rotary drive element and said other components are moveable axially between an engaged position in which the cooperating elements engage to provide said tactile and/or audible signal upon rotation of said drive element, and a disengaged position.

16. An injection device comprising;
a housing (10,16) receiving a cartridge or syringe (18);
a plunger (50) moveable axially to express a dose from said cartridge or syringe, and
an arrangement for emitting a series of mechanically generated tactile and/or audible signals during at least part of the movement of said plunger.

17. An injection device according to claim 2, wherein said clutch member (38) is operable in said control position to effect at least one of reduction of angular velocity and interruption of said rotary movement.

18. An injection device according to claim 2, including an externally accessible trigger element (12) manually operable to move said clutch member (38) between said holding position, said release position and said control position.

19. An injection device according to claim 18, wherein said trigger element is moveable axially to effect operation of said clutch member.
